(19) United States
(12) Patent Application Publication
(10) Pub. No.: US 2020/0289749 A1
(43) Pub. Date: Sep. 17, 2020

(54) DROPLET MEASUREMENTSYSTEM, DROPLET MEASUREMENT METHOD AND DROPLET MEASUREMENT PROGRAM

(71) Applicants: ICOMES LAB., Co., Ltd., Morioka City, Iwate (JP); IGUNOSS, Inc., Kitakami City, Iwate (JP)

(72) Inventors: Naoyuki ODASHIMA, Morioka City, Iwate (JP); Tadataka KAMIYAMA, Morioka City, Iwate (JP); Isao OHWADA, Kitakami City, Iwate (JP); Harumi SANGAWA, Kitakami City, Iwate (JP); Ryoichi AKAI, Osaka City (JP)

(21) Appl. No.: 15/775,088

(22) PCT Filed: Nov. 11, 2016
(86) PCT No.: PCT/JP2016/083485

§ 371 Date: May 10, 2018

(30) Foreign Application Priority Data
Nov. 13, 2015 (JP) 2015-223503

(51) Int. Cl. A61M 5/168; A61M 5/14; A61M 39/28

(52) U.S. Cl. CPC A61M 5/1689; A61M 5/1411; A61M 5/16804; A61M 2205/3379; A61M 2205/3334; A61M 2205/583; A61M 2205/50; A61M 39/28

(57) ABSTRACT

A droplet measurement system, etc. is provided that is capable of measuring the volume of a droplet with high precision by means of image processing with a lighter arithmetic load than before. The droplet measurement system is a droplet measurement system that measures the volume of a droplet dripping from a nozzle being provided with: an imaging unit that images an object to be imaged and outputs image data, wherein the imaging unit is placed such that a field of view is directed to a tip of the nozzle and a region extending vertically downward from the tip; an imaging control unit that sets the field of view of the imaging unit to a rectangular region long in a vertical direction and that causes the imaging unit to execute imaging; and an image processing unit that: acquires a plurality of images, in a temporal sequence order, that correspond to the rectangular region and that have captured therein the tip of the nozzle and the region extending vertically downward from the tip based on the image data output from the imaging unit; and that calculates the volume of the droplet based on the images acquired in a temporal sequence order.

### TECHNICAL FIELD

The present invention relates to a droplet measurement system that measures the volume of droplets dripping down from a nozzle, a droplet measurement method and a droplet measurement program.

### BACKGROUND ART

It is important to maintain a predetermined flow rate when delivering liquid (infusion liquid), such as chemicals, nutritional supplements, or the like, intravenously. Conventionally, the infusion flow rate control has been performed by determining the number of drips per unit time by counting the number of droplets dripped in a drip tube, calculating the flow rate based on an assumption that the droplet volume is constant, and adjusting the dripping cycle of the droplets (i.e. the time interval of dripping).

However, in practice, the surface tension of the infusion liquid varies depending on the conditions, such as viscosity, ambient temperature, or the like, and thus, the volume per droplet is not constant. Moreover, in medical settings, patients may change their positions during infusion, and in such case, the head difference of the infusion liquid may change, and the volume of droplets may vary. Accordingly, the conventional method that controls the flow rate solely based on the dripping cycle of the droplets has been prone to errors in flow rate, and high-precision flow rate control has been difficult.

To handle such problems, a technique is known in which the volume of the dripping droplets is measured and used in the flow rate control. For example, PTL 1 discloses a droplet detection device provided with: a transparent drip tube; a light emitting part arranged on one side of the exterior of the drip tube; and a two-dimensional image sensor arranged at a position opposite to the light emitting part with the drip tube sandwiched therebetween, wherein a field of view of the two-dimensional image sensor is set such that a tip of a dripping nozzle in the drip tube and a predetermined droppage distance of droplets dropping from the dripping nozzle are included.

### PRIOR ART DOCUMENTS

### Patent Literature

### PTL 1: JP2011-62371 A

### SUMMARY OF THE INVENTION

### Problems to be Solved by the Invention

In PTL 1, the state in which a droplet separates and drops from the dripping nozzle tip is imaged at predetermined time intervals, an image that is one before the imaged image is investigated when the state of the droplet in such imaged image is determined to be the “droplet-dropped-state,” and the volume of the droplet is calculated from such image determined to be in the “droplet-dropped-state”when the state of the droplet in such image that is one before the imaged image is determined to be the “state before droplet droppage.”

In the case of PTL 1, the processing is cumbersome, and since image processing for detecting the droplet state needs to be performed on all imaged images and thus, the operational load is heavy. In order to cope with this, a high-performance processor is necessary and, as a result, the miniaturization or simplification of the entire droplet measurement system becomes difficult.

The present invention has been made in view of the above-described problems, and an object thereof is to provide a droplet measurement system, a droplet measurement method and a droplet measurement program, which are capable of measuring the droplet volume with high precision by means of image processing with a lighter arithmetic load than before.

### Means for Solving the Problems

In order to solve the above-described problems, a droplet measurement system according to an aspect of the present invention is a droplet measurement system that measures the volume of a droplet dripping from a nozzle being provided with: an imaging unit that images an object to be imaged and outputs image data, wherein the imaging unit is placed such that a field of view is directed to a tip of the nozzle and a region extending vertically downward from the tip; an imaging control unit that sets the field of view of the imaging unit to a rectangular region long in a vertical direction and that causes the imaging unit to execute imaging; and an image processing unit that: acquires a plurality of images, in a temporal sequence order, that correspond to the rectangular region and that have captured therein the tip of the nozzle and the region extending vertically downward from the tip based on the image data output from the imaging unit; and that calculates the volume of the droplet based on the images acquired in a temporal sequence order.

In the above-described droplet measurement system, the image processing unit may include: a liquid discontinuity detection unit that detects a liquid discontinuity image that is an image that has captured therein the state immediately after the droplet separated from the tip, from the images in a temporal sequence order; and a volume calculation unit that: acquires the liquid discontinuity image and a predetermined number of images following the liquid discontinuity image from the images in a temporal sequence order; and that calculates the volume using the liquid discontinuity image and the predetermined number of images.

The above-described droplet measurement system may be further provided with a sensor that detects a tilt of the nozzle with respect to an axis in the vertical direction, and the image processing unit may further include a correction unit that performs a correction operation for the volume calculated by the volume calculation unit based on the tilt detection result by the sensor.

In the above-described droplet measurement system, the image processing unit may further include a correction unit that retains a reference value of dimensions of an image of the nozzle in an image corresponding to the rectangular region and that performs a correction operation for the volume calculated by the volume calculation unit based on the reference value and dimensions of an image of the nozzle captured in the liquid discontinuity image or the predetermined number of images.

In the above-described droplet measurement system, an imaging frame rate of the imaging unit may be 100 frames per second or more.

In the above-described droplet measurement system, a ratio of the length in the vertical direction in the rectangular region to the length in a horizontal direction thereof may be from 1.5 to 4.5, inclusive.

In the above-described droplet measurement system, the nozzle may be provided to an infusion device that performs infusion of liquid filled in a container via a drip tube and the nozzle drips the droplet consisting of the liquid in the drip tube, and the droplet measurement system may further be provided with: an actuator that varies a flow rate of the liquid by varying a pressing force with respect to a tube that causes the liquid accumulated in the drip tube to flow therethrough by driving a clamp that is provided on the tube in a pressable manner; and a flow rate control unit that controls the actuator such that the flow rate of the liquid falls within a predetermined range based on the volume calculation result.

In the above-described droplet measurement system, the nozzle may be provided to an infusion device that performs infusion of liquid filled in a container via a drip tube and the nozzle may drip the droplet consisting of the liquid in the drip tube, and the droplet measurement system may further be provided with: an actuator that varies a flow rate of the liquid by varying a pressing force with respect to a tube that causes the liquid accumulated in the drip tube to flow therethrough by driving a clamp that is provided on the tube in a pressable manner; a storage unit that stores therein information that is acquired in advance and that represents correlation between the volume of the droplet and a dripping cycle of the droplet; and a flow rate control unit that controls the actuator such that the flow rate of the liquid falls within a predetermined range based on the information.

In the above-described droplet measurement system, the storage unit may store therein multiple types of the information according to liquid types, and the flow rate control unit may acquire information corresponding to the liquid filled in the container from the multiple types of the information stored in the storage unit and control the actuator based on the acquired information.

The above-described droplet measurement system may further be provided with: a light source that is provided opposite to the imaging unit and that illuminates at least the tip of the nozzle and the region extending vertically downward from the tip; and a filter that controls light distribution of light output from the light source.

In the above-described droplet measurement system, the imaging unit may further include a telecentric lens.

A droplet measurement method according to an aspect of the present invention is a droplet measurement method of measuring the volume of a droplet dripping from a nozzle, including: an imaging control step of setting a field of view of an imaging unit to a rectangular region long in a vertical direction and causing the imaging unit to execute imaging, wherein the imaging unit images an object to be imaged and outputs image data, and wherein the imaging unit is placed such that the field of view is directed to a tip of the nozzle and a region extending vertically downward from the tip; and an image processing step of acquiring a plurality of images, in a temporal sequence order, that correspond to the rectangular region and that have captured therein the tip of the nozzle and the region extending vertically downward from the tip, based on the image data output from the imaging unit, and calculating the volume of the droplet based on the images acquired in a temporal sequence order.

A droplet measurement program according to an aspect of the present invention is a droplet measurement program for measuring the volume of a droplet dripping from a nozzle, causing a computer to execute: an imaging control step of setting a field of view of an imaging unit to a rectangular region long in a vertical direction and causing the imaging unit to execute imaging, wherein the imaging unit images an object to be imaged and outputs image data, and wherein the imaging unit is placed such that the field of view is directed to a tip of the nozzle and a region extending vertically downward from the tip; and an image processing step of acquiring a plurality of images, in a temporal sequence order, that correspond to the rectangular region and that have captured therein the tip of the nozzle and the region extending vertically downward from the tip, based on the image data output from the imaging unit, and calculating the volume of the droplet based on the images acquired in a temporal sequence order.

### Effect of the Invention

Since the present invention sets the field of view of the imaging unit to a rectangular region long in the vertical direction and calculates the droplet volume based on the images, in a temporal sequence order, that have imaged therein the tip of the nozzle and the region extending vertically downward from the tip by means of the imaging unit, the droplet volume can be measured with high precision by means of image processing with a lighter arithmetic load than before.

### BRIEF DESCRIPTION OF THE DRAWINGS

FIG. 1 is a diagram showing a schematic configuration of a droplet measurement system according to a first embodiment of the present invention.

FIG. 2 is a block diagram showing a schematic configuration of an arithmetic device shown in FIG. 1.

FIG. 3 is a schematic diagram for describing a field of view of an imaging element shown in FIG. 1.

FIG. 4 is a schematic diagram showing an example of a screen displayed on a display device during the operation of the droplet measurement system shown in FIG. 1.

FIG. 5 is a flowchart illustrating the operation of the droplet measurement system shown in FIG. 1.

FIG. 6 is a flowchart illustrating the details of the droplet volume calculation shown in FIG. 5.

FIG. 7 is a schematic diagram showing an example of an image generated by an image processing unit shown in FIG. 2.

FIG. 8 is a diagram showing a schematic configuration of a droplet measurement system according to a second embodiment of the present invention.

FIG. 9 is a block diagram showing a schematic configuration of an arithmetic device shown in FIG. 8.

FIG. 10 is a schematic diagram for describing a correction operation in the second embodiment of the present invention.

FIG. 11A is a schematic diagram for describing a correction operation in a third embodiment of the present invention.

FIG. 11B is a schematic diagram for describing a correction operation in the third embodiment of the present invention.

FIG. 12A is a schematic diagram for describing a correction operation in the third embodiment of the present invention.

FIG. 12B is a schematic diagram for describing a correction operation in the third embodiment of the present invention.

FIG. 13 is a graph showing an example of correlation between a dripping cycle of droplets and the volume per droplet in the infusion.

FIG. 14 is a block diagram showing a schematic configuration of an arithmetic device provided to a droplet measurement system according to a fourth embodiment of the present invention.

FIG. 15 is a flowchart illustrating the operation of the droplet measurement system according to the fourth embodiment of the present invention.

### EMBODIMENTS OF THE INVENTION

The droplet measurement system, the droplet measurement method and the droplet measurement program according to embodiments of the present invention will be described hereinafter, with reference to the drawings. It should be noted that the present invention is not limited by these embodiments. In addition, in the descriptions of the respective drawings, identical parts are denoted by identical reference numbers.

### First Embodiment

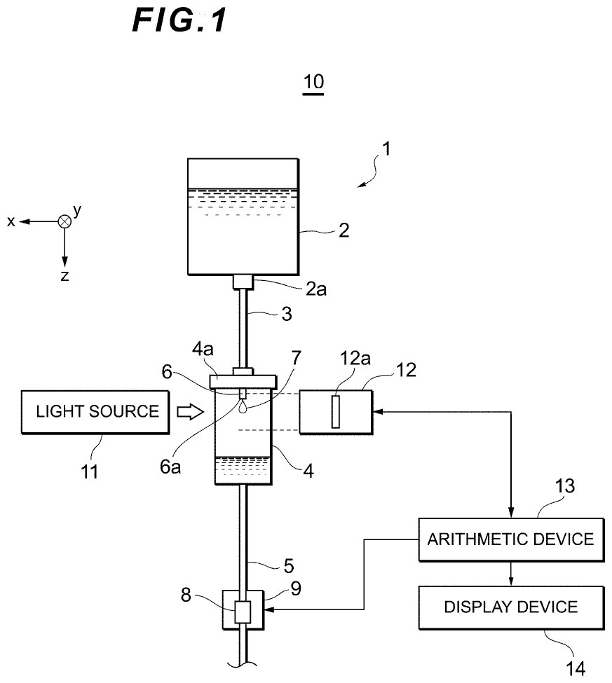

FIG. 1 is a diagram showing a schematic configuration of a droplet measurement system according to a first embodiment of the present invention. As shown in FIG. 1, the droplet measurement system 10 according to the present embodiment is a system that measures the volume of droplets 7 dripping down from a tip 6a of a nozzle 6 (hereinafter also referred to as a nozzle tip) provided inside a drip tube 4 and that controls the flow rate of the infusion based on the measured volume, with respect to an infusion device 1 that supplies liquid (infusion liquid) filled in an infusion bag 2 via an intermediate tube 3, a drip tube 4 and an infusion tube 5.

The infusion bag 2 is a container filled with infusion liquid, such as chemicals, nutritional supplements, or the like, and is held in a suspended manner from a support, etc. during infusion. The intermediate tube 3 is connected, at one end thereof, to a drainage port 2a of the infusion bag 2 and is connected, at the other end thereof, to one end of the nozzle 6 that is attached to an upper lid 4a of the drip tube 4. The other end of this nozzle 6 is provided such as to project into the drip tube 4.

The infusion tube 5 is made of an elastic material. A clamp 8 that is capable of pressing the infusion tube 5 in a radial direction and an actuator 9 that drives the clamp 8 are provided in the midway of the infusion tube 5.

The actuator 9 varies the pressing force exerted by the clamp 8 with respect to the infusion tube 5 by driving the clamp 8 under the electrical control. By means of which the inner diameter of the infusion tube 5 changes (opens and closes), and then the flow rate of the infusion liquid that flows in the infusion tube 5 can be adjusted. Along with this, the internal pressure of the drip tube 4 changes and thus, the dripping cycle of the droplets 7 dripping down from the nozzle 6 changes.

The droplet measurement system 10 is further provided with a light source 11 that illuminates the drip tube 4, an imaging unit 12 that images the interior of the drip tube 4 and generates image data, an arithmetic device 13 that calculates the volume of the droplets based on the image data generated by the imaging unit 12, and a display device 14 that displays the result of the droplet volume calculations, or the like.

The light source 11 is provided with a light emitting element, for example, a light emitting diode (LED) or the like, and an optical system, such as a filter, a lens or the like, that controls the light distribution such that the light output from the light emitting element becomes parallel light. The light source 11 is placed opposite to the field of view of the imaging unit 12 and illuminates at least the nozzle tip 6a, from which the droplets drip down, and the region including the vertically lower side of the nozzle tip 6a, from behind the droplets 7.

The imaging unit 12 is a camera capable of high-speed imaging and is provided with an imaging element 12a consisting of a charge coupled device (CCD) image sensor, a complementary metal oxide semiconductor (CMOS) image sensor or the like, and the imaging unit 12 outputs image data after imaging an object to be imaged. It is preferable for the imaging frame rate to be set to 100 frames per second (fps) or higher, and it is set to 120 fps in the present first embodiment.

The specifications of the imaging unit 12 can be configured as appropriate, depending on the infusion device 1 being the target of measurement. As an example, when the infusion device 1 is a device generally used in the medical field, a compact camera may be used with the size of the outer diameter of a camera module ranging from approximately a few millimeters to several dozens of millimeters and with the focus distance thereof ranging from a few millimeters to a few tens of millimeters, preferably approximately several dozens of millimeters, such that the drip tube 4 can be imaged at close range and also such that the infusion operation by a user is not hindered.

Preferably, a telecentric lens may be provided on the imaging unit 12, by means of which, variations in the image size of the droplet 7 caused by the change in the distance between the droplet 7 and the imaging element 12a can be suppressed and thus, the error in volume calculation of the droplet 7 can be suppressed.

As for the imaging element 12a, an imaging element having a general geometry with an aspect ratio of a light-receiving surface being 1 (long): 2 (wide) or 2:3 may be used. However, as described below, at the orientation at which the imaging unit 12 is placed during, at least, the operation of the droplet measurement system 10, the imaging unit 12 is controlled such that the rectangular region of the light-receiving surface of the imaging element 12a, which is long in the vertical direction, becomes the effective imaging region that actually takes in the images. Namely, image signals are acquired only from the pixels arranged in the effective imaging region. Here, the vertical direction refers to the direction in which the droplets 7 drip down (i.e. the z direction), namely, the direction of gravitational force. Needless to say, a rectangular element having a specific aspect ratio may be used as the imaging element 12a, the imaging unit 12 may be placed such that the long side of the imaging element 12a becomes the vertical direction, and the image signals may be acquired from pixels across the light-receiving surface of the imaging element 12a. By controlling the imaging region of the imaging element 12a in this manner, the field of view of the imaging unit 12 is set to the rectangular region that is long in the vertical direction.

Such imaging unit 12 is placed such that the nozzle tip 6a and the region extending vertically downward from the nozzle tip 6a coincide with the field of view. The aspect ratio of the field of view set for the imaging unit 12 (i.e. the imaging region of the imaging element 12a) will be described below.

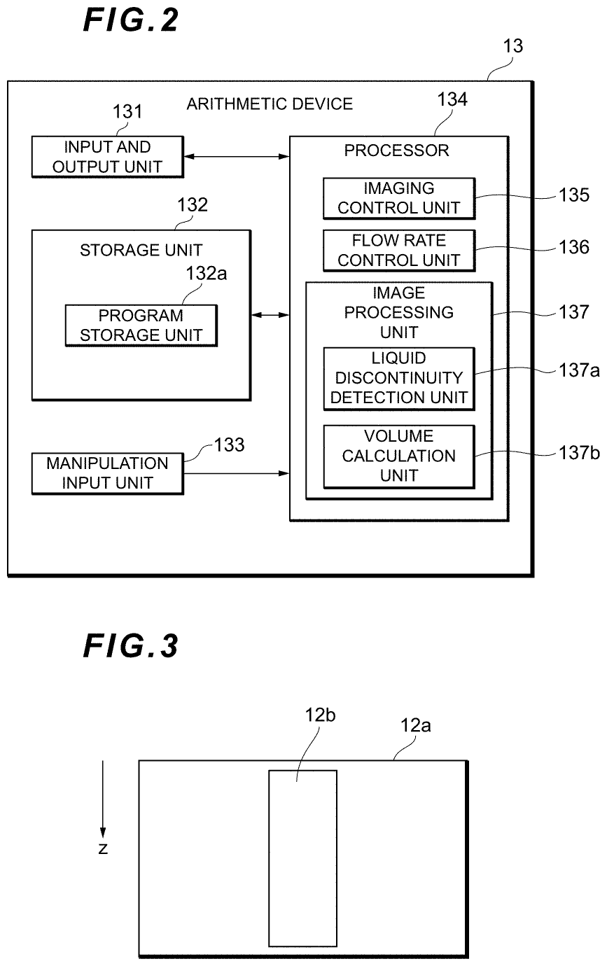

As for the arithmetic device 13, in addition to devices configured specifically for the present droplet measurement system 10, a general-purpose arithmetic device, such as a personal computer or the like, may be used. FIG. 2 is a block diagram showing the schematic configuration of the arithmetic device 13. As shown in FIG. 2, the arithmetic device 13 is provided with an input and output unit 131, a storage unit 132, a manipulation input unit 133 and a processor 134.

The input and output unit 131 is an external interface that performs input and output of image data or various signals among various external devices, such as the imaging unit 12, the display device 14, and the like.

The storage unit 132 is configured by a disk drive, a semiconductor memory, such as ROM or RAM or the like. The storage unit 132 includes a program storage unit 132a that stores therein control programs for controlling various units of the arithmetic device 13 and programs that causes the arithmetic device 13 to execute predetermined operation. More specifically, the program storage unit 132a stores therein an image processing program for calculating the volume of the droplets from a plurality of images capturing the droplet based on the image data input from the imaging unit 12. In addition, the storage unit 132 stores therein various parameters to be used for execution of the image processing program, the image data input from the imaging unit 12 and the result of droplet volume calculation, or the like.

The manipulation input unit 133 is configured by an input device, such as an input button, a switch, a keyboard, a mouse, a touch panel or the like, and inputs signals according to the manipulations made by a user into the processor 134.

The processor 134 is configured by an arithmetic and logic unit, such as a CPU or the like, and various registers, and it performs data transfer and provides instructions to the respective units of the arithmetic device 13 by reading and executing the various programs stored in the program storage unit 132a, and integrally controls the operation of the arithmetic device 13.

More specifically, the processor 134 includes: an imaging control unit 135 that controls the operation of the imaging unit 12; a flow rate control unit 136 that controls the flow rate of the infusion liquid in the infusion device 1; and an image processing unit 137 that performs image processing, such as volume calculation or the like, of the droplet 7 based on the image data input from the imaging unit 12.

The imaging control unit 135 causes the imaging unit 12 to operate at a predetermined imaging frame rate and also performs the control to limit the imaging region of the imaging element 12a. More specifically, as shown in FIG. 3, the imaging control unit 135 sets the rectangular region of the light-receiving surface of the imaging element 12a, which is long in the vertical direction (the z-direction), as the effective imaging region 12b and controls the imaging element 12a such that image signals are acquired only from the pixels arranged in this imaging region 12b.

The aspect ratio of the imaging region 12b may be determined such that a few shots (three or more shots) of the state in which the droplets 7 are separating and falling from the nozzle tip 6a can be imaged, based on the relationship between the size of the droplet 7, which is determined based on the diameter of the nozzle 6 or the type of the infusion liquid (viscosity, etc.), and the imaging frame rate.

More specifically, the width in the horizontal direction of the image corresponding to the imaging region 12b may preferably be set to approximately 1.5 to 2 times the size of the image of droplet 7, in consideration of the disturbance when the droplet 7 is falling down. The length in the perpendicular direction of the image may preferably be set to approximately 3 to 9 times the size of the image of the droplet 7, in consideration of the falling velocity of the droplet 7. The reason for this is that, when the length in the perpendicular direction is too short, the falling droplet 7 will go out of sight immediately, and when it is too long, the image of the accelerated droplet 7 will be blurred at the lower part of the image, and thus, in any of the cases, such imaging region is not appropriate for use in the image processing.

Accordingly, the aspect ratio of the imaging region 12b, i.e. the ratio of the longitudinal (long side) to latitudinal (short side) may be within a range of approximately 1.5 to 4.5, inclusive. The specific size examples of the imaging region 12b include 1936 pixels long×1096 pixels wide, 1936 pixels long×496 pixels wide, and the like.

The flow rate control unit 136 controls the operation of the actuator 9 based on the volume of the droplet 7 calculated by the image processing unit 137. Here, the flow rate of the infusion liquid is obtained by dividing the volume of the droplet 7 by the dripping cycle of the droplets 7. The flow rate control unit 136 retains therein a target flow rate predetermined by a user and performs control such that the actual flow rate approaches this target flow rate.

The image processing unit 137 generates, in a temporal sequence order, vertically long rectangular images that have captured therein the nozzle tip 6a and the region extending vertically downward from the nozzle tip 6a, based on the image data input from the imaging unit 12, and performs processing for calculating the volume of the droplet 7 based on these images. More specifically, the image processing unit 137 is provided with a liquid discontinuity detection unit 137a and a volume calculation unit 137b.

The liquid discontinuity detection unit 137a detects an image that has captured therein the state immediately after the droplet 7 has separated from the nozzle tip 6a (i.e. a liquid discontinuity image) from the plurality of images generated in a temporal sequence order. The volume calculation unit 137b acquires the liquid discontinuity image and a predetermined number of images that follow the liquid discontinuity image in a temporal sequence order, and calculates the volume of the droplet that is captured in the acquired images by performing predetermined image processing on these images.

The display device 14 is configured by a liquid crystal display, an organic EL display, or the like, and displays a predetermined screen based on control signals output from the arithmetic device 13 and image data for display, under the control of the arithmetic device 13.

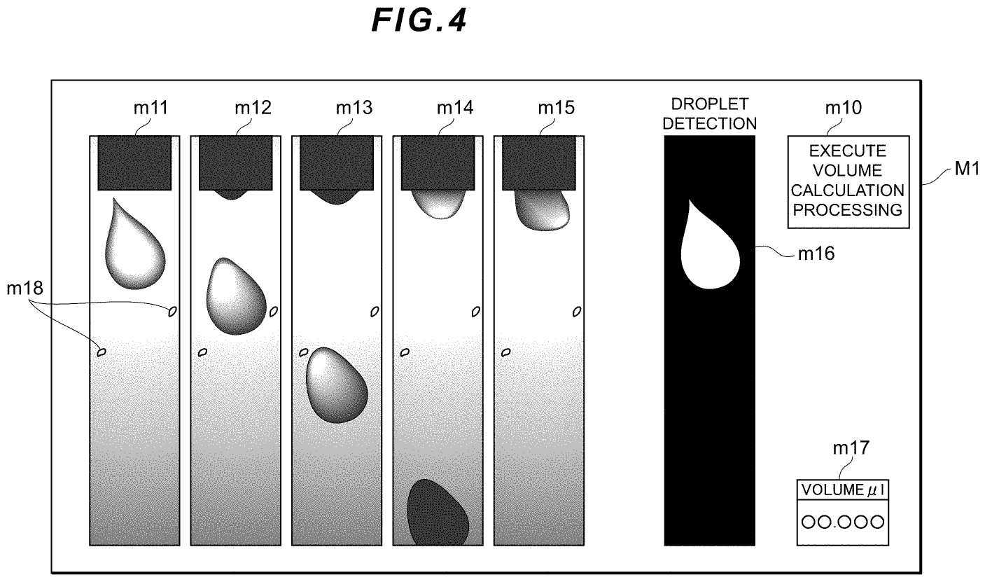

FIG. 4 is a schematic diagram showing an example of a screen displayed on the display device 14 during the operation of the droplet measurement system 10. As shown in FIG. 4, the screen M1 includes: an instruction button m10 for inputting an instruction for executing the volume calculation processing of the droplet 7; a plurality of images m11 to m15 generated based on the image data input from the imaging unit 12; an image m16 that shows the two dimensional shape of the droplet 7 detected by the image processing on these images m11 to m15; and a display field m17 where the volume calculation result of the droplet 7 is displayed.

It should be noted that, in the droplet measurement system 10, the display device 14 is not required and the images m11 to m16 shown in FIG. 4 also need not be displayed on the screen. In addition, instead of the display device 14, a compact display device that only shows the volume calculation result of the droplet 7 in numerical values may be provided, a compact display device that only shows the actual flow rate, in numerical values, that is controlled based on the volume calculation result may be provided, or a display device that displays an alarm based on the volume calculation result may be provided. Moreover, an audio generation device, such as a speaker or the like, that informs the alarm through voice or a particular sound may also be added.

The imaging unit 12, the arithmetic device 13 and the display device 14 may be provided as separate devices that are connected to each other by a cable, or these devices may be accommodated in a single housing. In the latter case, since the droplet measurement system 10 can be configured by: the housing in which the imaging unit 12, the arithmetic device 13 and the display device 14 are accommodated; and the light source 11, the entire droplet measurement system 10 can be miniaturized and it is also easy to carry around.

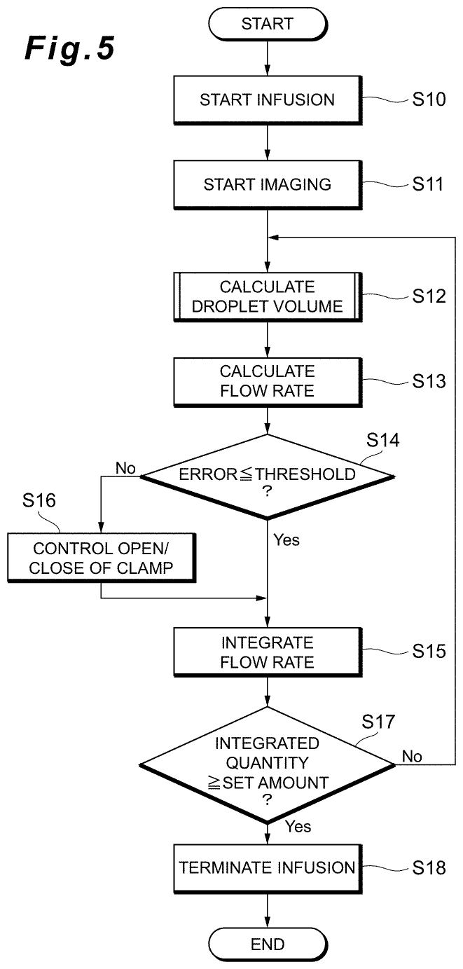

Next, the operation of the droplet measurement system 10 will be described. FIG. 5 is a flowchart illustrating the operation of the droplet measurement system 10.

Before starting the infusion, a user places the light source 11 and the imaging unit 12 in the neighborhood of the drip tube 4 (see FIG. 1). At this time, the positional relationship among the light source 11, the drip tube 4 and the imaging unit 12 may be adjusted, while looking at the screen displayed on the display device 14 (see FIG. 4), such that the nozzle tip 6a and the region extending vertically downward from the nozzle tip 6a are located within the field of view of the imaging element 12a.

In step S10, the infusion operation in the infusion device 1 starts by driving the actuator 9, under the control of the flow rate control unit 136, to cause the clamp 8 to open the infusion tube 5.

In the subsequent step S11, the imaging control unit 135 causes the imaging unit 12 to start imaging at a predetermined imaging frame rate at the predetermined imaging region 12b (see FIG. 3).

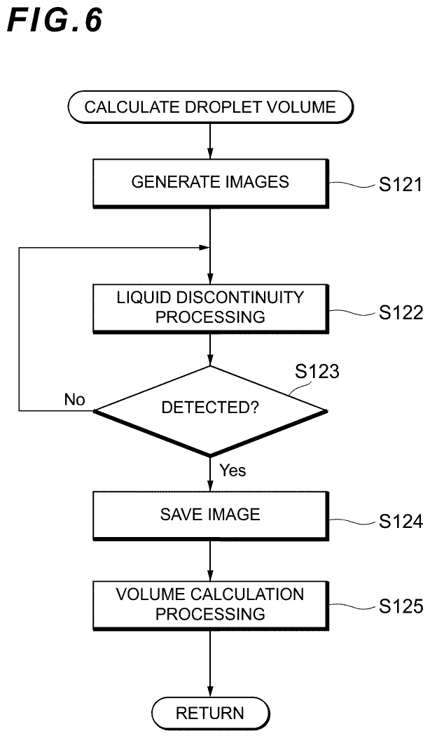

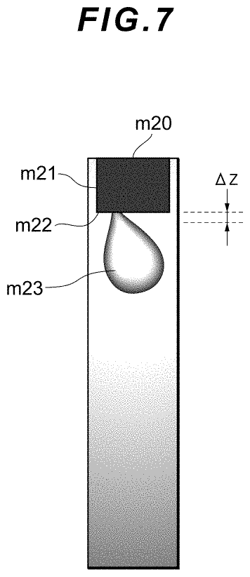

In the subsequent step S12, the image processing unit 137 calculates the volume of a droplet 7. FIG. 6 is a flowchart illustrating the details of the volume calculation of the droplet 7. FIG. 7 is a schematic diagram showing an example of an image generated by the image processing unit 137. The image m20 shown in FIG. 7 captures therein the respective images m21, m22, m23 of the nozzle 6, the nozzle tip 6a and the droplet 7 shown in FIG. 1.

First, in step S121, the image processing unit 137 generates images (luminance images) in a temporal sequence order by sequentially acquiring the image data output from the imaging unit 12 and by applying predetermined processing thereon.

In step S122, the image processing unit 137 executes liquid discontinuity detection processing on the generated images. More specifically, the liquid discontinuity detection unit 137a determines, with respect to the image (see, for example, image m20 in FIG. 7) generated in step S121, whether the luminance of each pixel contained a line within the predetermined range Δz directly below the image m22 of the nozzle tip 6a is equal to or larger than a threshold. When the luminance of all pixels contained in the line within the range Δz is equal to or larger than the threshold, such range Δz is determined to be the background. The liquid discontinuity detection unit 137a sequentially performs this determination on the generated images and detects an image in which the range Δz changed from the non-background state to the background state as the liquid discontinuity image.

When the liquid discontinuity image is not detected (step S123: No), the image processing unit 137 continues to execute the liquid discontinuity detection processing on the sequentially generated images (step S122). In this case, the images used for the previous liquid discontinuity detection processing may be deleted.

On the other hand, when the liquid discontinuity image is detected (step S123: Yes), the image processing unit 137 saves the liquid discontinuity image (see image m11 in FIG. 4) and a predetermined number of images following this liquid discontinuity image (see images m12 to m15 in FIG. 4) on a memory (step S124). At this time, the saved images m11 to m15 may be displayed on the display device 14 as shown in FIG. 4.

At this time, the image processing unit 137 calculates the image interval between the currently-detected liquid discontinuity image and the previously-detected liquid discontinuity image, and calculates the dripping cycle of the droplets 7 by multiplying the image interval and the imaging frame rate.

In the subsequent step S125, the image processing unit 137 executes the volume calculation processing of the droplet 7. More specifically, the volume calculation unit 137b performs an operation by a predetermined algorithm using the images m11 to m15 saved in step S124. As for the algorithm, various publicly-known approaches may be used. As an example, noise m18 may be removed based on the saved images, only the image of the moving (falling) droplet 7 may be extracted as a target to be measured, and the two-dimensional shape may be detected by threshold processing. The volume of the droplet 7 is calculated based on this two-dimensional shape.

It should be noted that five images m11 to m15 are shown in FIG. 4 as images saved on a memory; however, the number of images to be saved is not limited to five, and the number necessary depending on the volume calculation processing algorithm of the droplet 7 may be saved as needed. However, the longer the time elapses from the beginning of the falling of the droplet 7, the blurrier the image of the droplet 7 gets by being accelerated, and thus, the image becomes inappropriate for image processing. Therefore, regarding the upper limit of the number of images, when the imaging frame rate is 120 fps, eight images to be saved is sufficient.

The image processing unit 137 also outputs the calculated droplet volume and causes the display device 14 to display the same. Thereafter, the processing returns to the main routine.

With reference to FIG. 5 again, in step S13 following step S12, the flow rate control unit 136 calculates the current flow rate of the droplet 7 (infusion liquid) by dividing the volume of the droplet 7 calculated in step S12 by the dripping cycle.

In step S14, the flow rate control unit 136 determines whether the error between the current flow rate calculated in step S13 and the predetermined target flow rate is equal to or less than a threshold. This threshold may be predetermined according to the purpose of infusion. It should be noted that, at this point, a determination may be made as to whether the current flow rate and the target flow rate are equal.

When the error is determined to be equal to or less than the threshold (step S14: Yes), the flow rate control unit 136 integrates the current flow rate in a memory (step S15). Thereby, the integrated quantity of the flow rate will be updated.

On the other hand, when the error between the current flow rate and the target flow rate is determined to be larger than the threshold (step S14: No), the flow rate control unit 136 performs opening/closing control of the clamp 8 via the actuator 9 (step S16). In particular, when the current flow rate is larger than the target flow rate, control for closing the clamp 8 is performed and when the current flow rate is smaller than the target flow rate, control for opening the clamp 8 is performed. Thereafter, the processing proceeds to step S15.

In step S17 following step S15, the flow rate control unit 136 determines whether the integrated quantity of the flow rate is equal to or larger than a predetermined flow rate set value. When the integrated quantity of the flow rate is determined to fail to satisfy the set value (step S17: No), the processing returns to step S12.

On the other hand, when the integrated quantity of the flow rate is determined to be equal to or larger than the set value (step S17: Yes), the flow rate control unit 136 terminates the infusion (step S18) by causing the clamp 8 to block the infusion tube 5 via the actuator 9. Thereafter, the imaging control unit 135 causes the imaging unit 12 to shut off the imaging operation. Accordingly, the operation of the droplet measurement system 10 is terminated.

As described above, since the first embodiment of the present invention performs the imaging exclusively with respect to the nozzle tip 6a and the region extending vertically downward from the nozzle tip 6a, the load required: for image data transmission from the imaging unit 12 to the arithmetic device 13; for image data transfer within the arithmetic device 13; and even for image processing executed by the image processing unit 137, can be reduced more than before. In addition, in the image processing unit 137, the liquid discontinuity detection processing is performed on the images generated in a temporal sequence order and the volume of the droplet 7 is calculated by means of the image processing using only the detected liquid discontinuity image and a predetermined number of images following the detected liquid discontinuity image and thus, high-precision volume calculation processing can be performed in real time with a lighter load than before. Accordingly, a high-performance processor can be dispensed with, and the miniaturization, simplification and even cost reduction of the configuration of the entire droplet measurement system can be achieved.

In addition, since the first embodiment of the present invention feed-back controls the flow rate via the actuator 9 based on the volume calculation result of the droplet 7, precise infusion can be performed.

### Second Embodiment

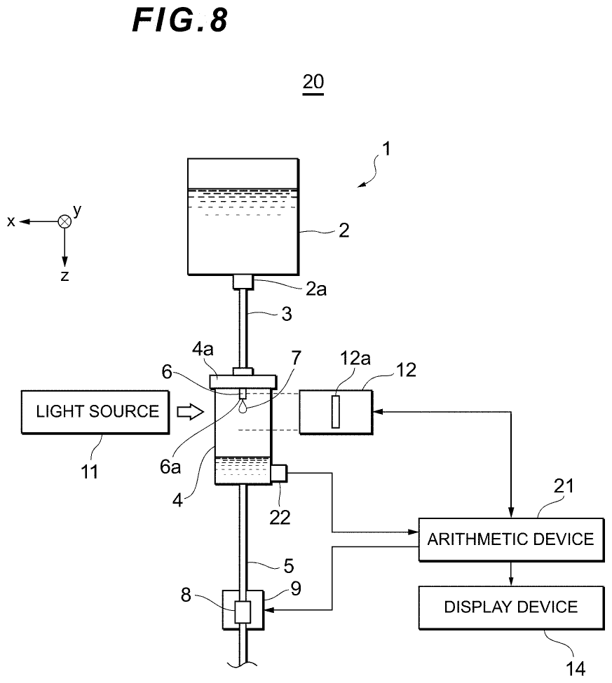

FIG. 8 is a diagram showing the schematic configuration of the droplet measurement system according to a second embodiment of the present invention. As shown in FIG. 8, the droplet measurement system 20 according to the second embodiment is obtained by providing an arithmetic device 21, instead of the arithmetic device 13, to the droplet measurement system 10 shown in FIG. 1 and it is further provided with a tilt sensor 22 attached to the drip tube 4. The configuration of the respective units of the droplet measurement system 20 other than the arithmetic device 21 and the tilt sensor 22 is similar to that of the first embodiment.

The tilt sensor 22 is configured by, for example, a gyroscope sensor or an acceleration sensor and detects the tilt of the drip tube 4 with respect to the axis in the vertical direction. Here, the nozzle 6 is fixed to the drip tube 4 and thus, the tilt of the drip tube 4 is substantially equal to the tilt of the nozzle 6.

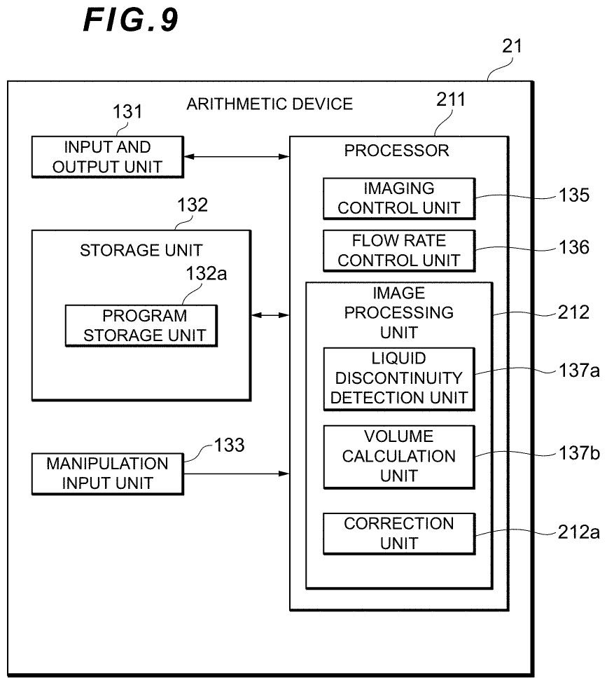

FIG. 9 is a block diagram showing the schematic configuration of the arithmetic device 21. As with the arithmetic device 13 shown in FIG. 2, the arithmetic device 21 is provided with an input and output unit 131, a storage unit 132 and a manipulation input unit 133, and only the configuration of a processor 211 differs from that of the processor 134 shown in FIG. 2. The processor 211 is provided with the imaging control unit 135, the flow rate control unit 136 and an image processing unit 212 which includes a correction unit 212a, in addition to the liquid discontinuity detection unit 137a and the volume calculation unit 137b. The operation of the imaging control unit 135, the flow rate control unit 136, the liquid discontinuity detection unit 137a and the volume calculation unit 137b is similar to that of the first embodiment.

The correction unit 212a performs a correction operation for the droplet volume calculation processing by the volume calculation unit 137b based on the tilt of the drip tube 4 (i.e. the tilt of the nozzle 6) detected by the tilt sensor 22.

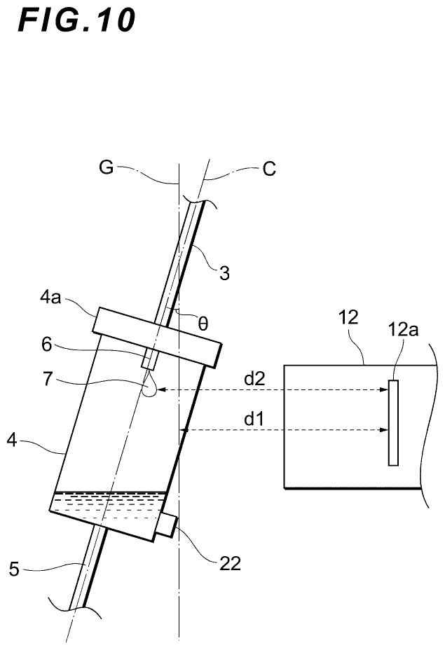

FIG. 10 is a schematic diagram for describing the correction operation in the second embodiment of the present invention and shows the state in which the center axis C of the drip tube 4 is tilted by an angle θ with respect to the axis G in the vertical direction. Here, if it is assumed that the drip tube 4 is not tilted, when the distance between the droplet 7, which is the object to be imaged, and the imaging element 12a is set to d1, the distance between the droplet 7 and the imaging element 12a will change to d2 due to the tilt of the drip tube 4. In other words, due to the variations in distances d1, d2 arising from the tilt of an angle θ, the image of the droplet 7 becomes enlarged or reduced, and thus, the precision of volume measurement is decreased.

Then, the correction unit 212a acquires the tilt angle θ detected by the tilt sensor 22 and corrects, after the liquid discontinuity detection processing (see step S122 in FIG. 6), the two-dimensional shape of the droplet (see image m16 in FIG. 4) detected by the volume calculation unit 137b in the volume calculation processing (see step S125 in FIG. 6) by way of enlargement or reduction in accordance with the angle θ. The volume calculation unit 137b calculates the volume of the droplet based on this corrected two-dimensional shape.

As described above, since the second embodiment of the present invention performs the correction operation based on the tilt of the drip tube 4 detected by the tilt sensor 22, higher precision volume calculation and flow rate control can be achieved.

### Third Embodiment

The configurations of the droplet measurement system according to the third embodiment of the present invention and the arithmetic device provided to such droplet measurement system are, on the whole, similar to those shown in FIGS. 8 and 9. In the present third embodiment, the correction unit 212a shown in FIG. 9 corrects the droplet volume calculation error arising from the focus deviation of the imaging unit 12 in the volume calculation processing (see step S125 in FIG. 6). In this case, the correction unit 212a may perform both the correction operation based on the tilt of the drip tube 4, as described in the second embodiment, and a correction operation described below, or the correction unit 212a may perform only the correction operation described below. In the latter case, the tilt sensor 22 shown in FIG. 8 may be omitted.

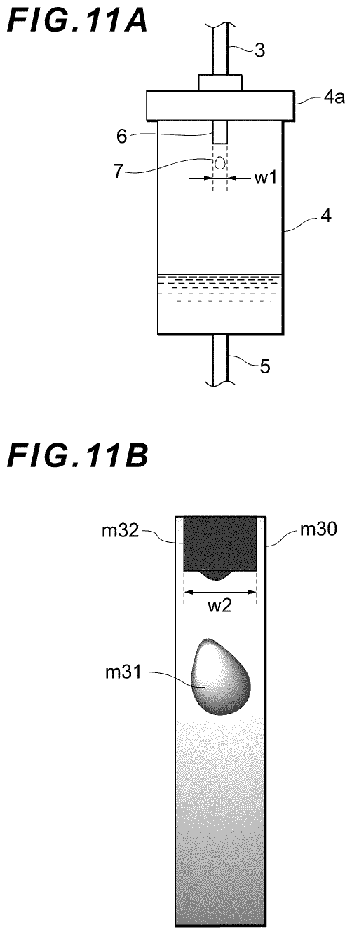

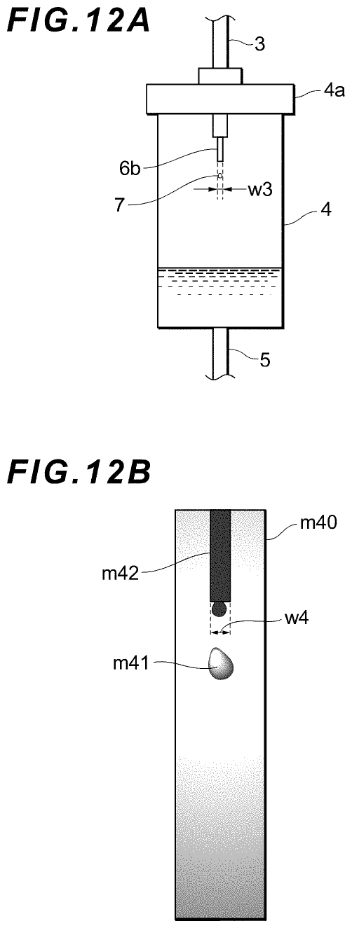

FIGS. 11A and 12B are schematic diagrams for describing the correction operation in the third embodiment of the present invention. For example, when performing a typical infusion of approximately 20 droplets/mL, the drip tube 4 having the nozzle 6 fixed to the upper lid 4a is used as shown in FIG. 11A. In this case, when measuring the volume of the droplet 7, imaging is performed by the imaging unit 12 focusing on the central axis of the nozzle 6. Typically, the nozzle 6 is designed such that its central axis coincides with the central axis of the drip tube 4 and thus, the relative position of the imaging unit 12 and the drip tube 4 is determined, using the position of the drip tube 4 as a guide, such that the imaging unit 12 focuses on the central axis of the drip tube 4. However, in practice, due to the component tolerance or the like, the position or tilt of the central axis of the nozzle 6 may deviate from the central axis of the drip tube 4. In such case, the focus of the imaging unit 12 deviates from the central axis of the nozzle 6 and the image m31 of the droplet 7 will be blurred in the image m30 shown in FIG. 11B. As a result, an error may be generated in the volume of the droplet 7 calculated based on such image m31.

For example, when performing a low flow rate infusion of approximately 60 droplets/mL, a separate needle (i.e. a needle-shaped nozzle) 6b is attached to and used with the drip tube 4 as shown in FIG. 12A. In this case, in addition to due to the component tolerance, the focus of the imaging unit 12 may deviate from the central axis of the needle 6b depending on how the needle 6b is attached. Accordingly, in the image m40 shown in FIG. 12B, the blurring of the image m41 of the droplet 7 may be generated.

Then, the correction unit 212a acquires, after the liquid discontinuity detection processing (see step S122 of FIG. 6), any of the images saved on the memory (see step S124 of FIG. 6) and corrects the droplet volume calculation error arising from the focus deviation of the imaging unit 12 based on the acquired image.

More specifically, the width of the image of the nozzle 6 or the image of the needle 6b on the image when the imaging unit 12 is focused on the central axis of the nozzle 6 or the needle 6b is calculated based on the relationship among the designed widths (diameters) w1, w3 of the nozzle 6 or the needle 6b, the focus length of the imaging unit 12 and the size of the imaging element 12a, and the calculated width is pre-retained in the correction unit 212a as a reference value.

When performing an infusion using the nozzle 6, the correction unit 212a calculates the ratio of the pre-retained reference value of the width of the image of the nozzle 6 and the width w2 of the image m32 of the nozzle 6 in the image m30 shown in, for example, FIG. 11B. Then, based on this ratio, the correction is made by way of enlargement or reduction of the two-dimensional shape of the droplet 7 (see image m16 of FIG. 4) detected by the volume calculation unit 137b. The volume calculation unit 137b calculates the volume of the droplet 7 based on this corrected two-dimensional shape.

When performing an infusion using the needle 6b, the correction unit 212a similarly calculates the ratio of the pre-retained reference value of the width of the image of the needle 6b and the width w4 of the image m42 of the needle 6b in the image m40 shown in, for example, FIG. 12B. Then, the two-dimensional shape of the droplet 7 is corrected using this ratio.

As described above, since the third embodiment of the present invention corrects the volume calculation error of the droplet 7 arising from the focus deviation of the imaging unit 12, higher precision volume calculation and flow rate control can be achieved.

### Fourth Embodiment

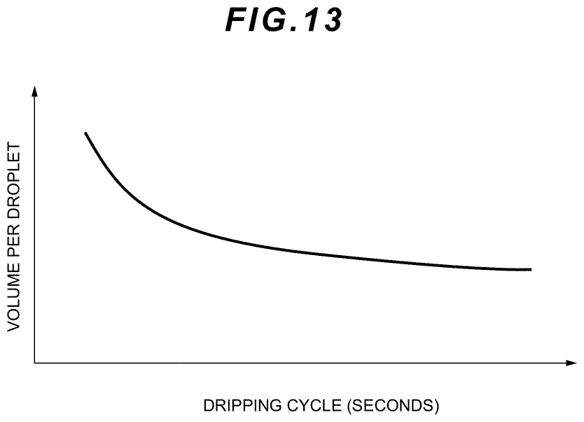

Next, the droplet measurement system according to a fourth embodiment of the present invention will be described. FIG. 13 is a graph showing an example of correlation between the dripping cycle (in seconds) of the droplets and the volume per droplet in the infusion, and is obtained through an experiment using the droplet measurement system 10 shown in FIG. 1.

Conventionally, the infusion flow rate control has been performed by adjusting the dripping cycle of the droplets based on the assumption that the volume of the droplets dripped down in the drip tube is constant. However, even when the dripping cycle was precisely controlled, in practice, the infusion still had not yet finished by the expected infusion termination time or, on the contrary, the infusion had already terminated, and thus, there existed a problem to the effect that high precision flow rate control was difficult.

Therefore, the inventors of the present application performed experiments of measuring the volume of the droplet 7 while changing the dripping cycle of the droplets 7 using the droplet measurement system 10 shown in FIG. 1. As a result, knowledge was obtained to the effect that the volume of the droplet 7 varied not only by the conditions, such as the viscosity of the infusion liquid, the environmental temperature or the like, or by the unexpected change in the situation, such as the movement of the patient, but also by the dripping cycle of the droplets 7. More specifically, as shown in FIG. 13, it was found that the volume per droplet tended to increase when the dripping cycle of the droplets was made shorter.

The present fourth embodiment utilizes the above-described knowledge and enables high precision flow rate control by acquiring in advance and accumulating, by means of the droplet measurement system 10 (or the droplet measurement system 20 shown in FIG. 8), the information representing the correlation between the dripping cycle of the droplets and the volume per droplet and by utilizing the accumulated information. The present fourth embodiment acquires the information representing the above-described correlation on an infusion liquid-type basis and enables the flow rate control according to the type of the infusion liquid.

Various methods are conceivable for the flow rate control based on the information representing the correlation between the dripping cycle of the droplets and the volume per droplet. In the present fourth embodiment, as an example, a method will be described in which the flow rate according to the dripping cycle is calculated based on the dripping cycle of the droplets and the volume per droplet, and a correlation table in which this flow rate and the dripping cycle are associated with each other is created and used in the flow rate control of the droplets. The flow rate according to the dripping cycle is calculated by dividing the droplet volume corresponding to the dripping cycle by the dripping cycle.

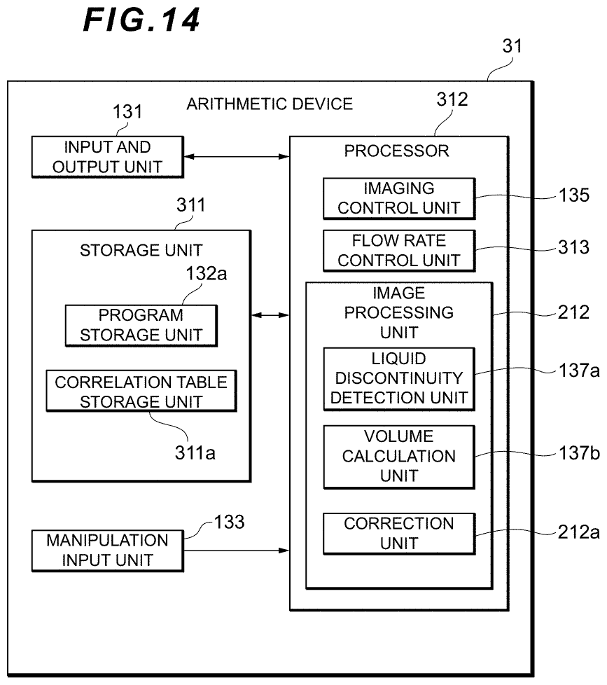

FIG. 14 is a block diagram showing the schematic configurations of the arithmetic device provided to the droplet measurement system according to the fourth embodiment of the present invention. It should be noted that the configuration of the droplet measurement system according to the fourth embodiment is, on the whole, common to that of FIG. 8.

As shown in FIG. 14, the arithmetic device 31 is provided with the input and output unit 131, a storage unit 311, the manipulation input unit 133 and a processor 312. Among these, the operation of the input and output unit 131 and the manipulation input unit 133 is similar to that of the first embodiment.

The storage unit 311 is provided with, in addition to the program storage unit 132a, a correlation table storage unit 311a that stores therein information representing the correlation between the dripping cycle of the droplets and the volume thereof in the form of a table. The correlation table storage unit 311a stores therein a plurality of such tables (correlation tables) on an infusion liquid-type basis. It should be noted that, as the information representing the correlation, a function may be used instead of the correlation table.

The processor 312 is provided with the imaging control unit 135, a flow rate control unit 313 and the image processing unit 212. Among these, the configuration and the operation of the imaging control unit 135 and the image processing unit 212 are similar to those of the first to third embodiments. It should be noted that, in the fourth embodiment, the tilt sensor 22 (see FIG. 8) and the correction unit 212a are not required and may therefore be omitted.

With reference to the correlation tables stored in the correlation table storage unit 311a, the flow rate control unit 313 acquires the dripping cycle adjustment amount necessary for making the current flow rate approach the target flow rate, and controls the actuator 9 based on this adjustment amount.

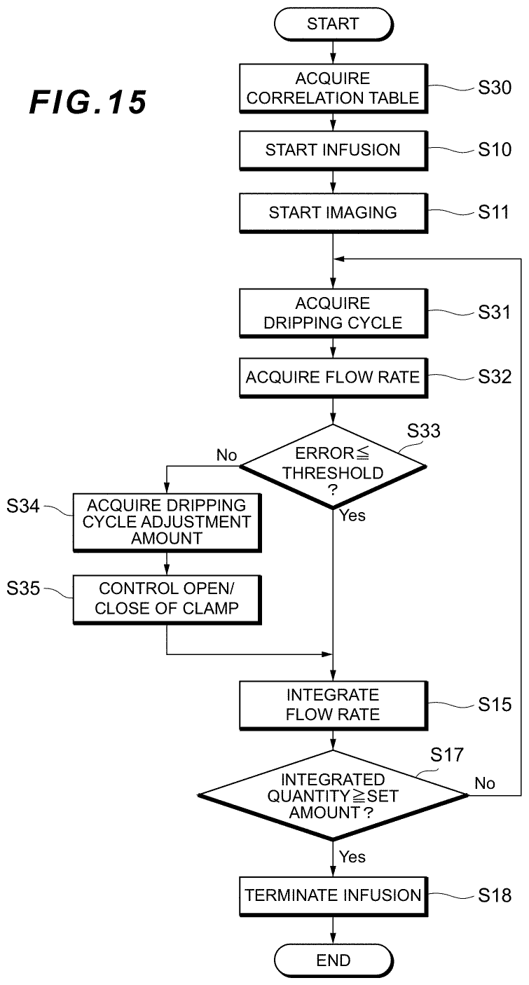

FIG. 15 is a flowchart illustrating the operation of the droplet measurement system according to the fourth embodiment.

Before starting the infusion, a user inputs information on the infusion liquid types into the arithmetic device 31 via the manipulation input unit 133. As with the first embodiment, the light source 11 and the imaging unit 12 are placed in the neighborhood of the drip tube 4.

In step S30, the flow rate control unit 313 acquires a correlation table associated with the infusion liquid type from the correlation table storage unit 311a based on the information input by the user in advance.

The processing in the subsequent steps S10, S11 is similar to that of the first embodiment.

In step S31 following step S11, the image processing unit 212 acquires the dripping cycle of the droplets 7. More specifically, the image processing unit 212 generates images in a temporal sequence order by sequentially acquiring the image data output from imaging unit 12 and by applying predetermined processing thereon, and executes liquid discontinuity detection processing on these images. It should be noted that the details of the liquid discontinuity detection processing are similar to those in step S122 in FIG. 6. After a liquid discontinuity image is detected, the image processing unit 212 calculates the image interval from the previously-detected liquid discontinuity image and calculates the dripping cycle by multiplying the image interval and the imaging frame rate.

In step S32, the flow rate control unit 313 acquires the flow rate (current flow rate) corresponding to the dripping cycle acquired in step S31 by referring to the correlation table.

In step S33, the flow rate control unit 136 determines whether the error between the current flow rate and the target flow rate is equal to or less than a threshold. It should be noted that, at this time, a determination may alternatively be made whether the current flow rate and the target flow rate are equal.

When the error is determined to be equal to or less than the threshold (step S33: Yes), the processing proceeds to step S15. The processing in step S15 and steps S17, S18, subsequent to step S15, is similar to that of the first embodiment.

On the other hand, when the error is determined to be larger than the threshold (step S33: No), the flow rate control unit 313 acquires the dripping cycle adjustment amount necessary for causing the current flow rate to transition to the target flow rate. More specifically, the flow rate control unit 313 acquires the dripping cycle corresponding to the target flow rate by referring to the correlation table and calculates the difference between such dripping cycle and the current dripping cycle. This difference corresponds to the dripping cycle adjustment amount.

In step S35, the flow rate control unit 136 performs opening and closing control of the clamp 8 via the actuator 9 by aiming at the dripping cycle adjustment amount acquired in step S34. For example, when the dripping cycle is made short, the volume of the droplet 7 becomes large and the flow rate increases suddenly and thus, the actuator 9 is controlled such that the clamp 8 is opened on a modest scale. Thereafter, the processing proceeds to step S15.

As described above, since the fourth embodiment of the present invention performs flow rate control by adjusting the dripping cycle based on the information representing the correlation between the pre-acquired dripping cycle of the droplets 7 and the volume thereof, the infusion liquid flow rate can be made to quickly approach the target flow rate.

The current dripping cycle is acquired by means of image processing in the above-described fourth embodiment; however, the current dripping cycle may be acquired by means of a conventional method of directly counting the number of dripping droplets by a sensor or the like. Namely, by combining the flow rate control based on the information representing the correlation as described in the present fourth embodiment with the conventional-type infusion system, desired flow rate control can be performed within a shorter time period and with higher precision than before.

The present invention is not limited to the above-described first to fourth embodiments and various inventions can be made by appropriately combining a plurality of components disclosed in the respective embodiments. For example, inventions can be made by removing certain components from the entirety of the components shown in the respective embodiments, or by appropriately combining the components shown in different embodiments.

### DESCRIPTION OF REFERENCE NUMBERS

1 Infusion device
2 Infusion bag
2a Drainage port
3 Intermediate tube
4 Drip tube
4a Upper lid
5 Infusion tube
6 Nozzle
6a Tip (nozzle tip)
6b Needle
7 Droplet(s)
8 Clamp
9 Actuator
10, 20 Droplet measurement system
11 Light source
12 Imaging unit
12a Imaging element
12b Imaging region
13, 21, 31 Arithmetic device
14 Display device
22 Tilt sensor
131 Input and output unit
132, 311 Storage unit
132a Program storage unit
133 Manipulation input unit
134, 211, 312 Processor
135 Imaging control unit
136, 313 Flow rate control unit
137, 212 Image processing unit
137a Liquid discontinuity detection unit
137b Volume calculation unit
212a Correction unit
311a Correlation table storage unit

1.-13. (canceled)

14. A droplet measurement system, comprising:
a nozzle that is provided to an infusion device that performs infusion of liquid filled in a container via a drip tube and that drips a droplet consisting of the liquid in the drip tube;
an actuator that varies a flow rate of the liquid by varying a pressing force with respect to a tube that causes the liquid accumulated in the drip tube to flow therethrough by driving a clamp that is provided on the tube in a pressable manner;
an acquisition unit that acquires a dripping cycle of droplets dripping from the nozzle;
a storage unit that stores therein information that is acquired in advance and that represents correlation between the volume of the droplet and the dripping cycle of the droplets; and
a flow rate control unit that controls the actuator such that the flow rate of the liquid falls within a predetermined range based on the information and the dripping cycle of the droplets dripping from the nozzle.

15. The droplet measurement system according to claim 14, wherein the storage unit stores therein multiple types of the information according to liquid types, and
wherein the flow rate control unit acquires information corresponding to the liquid filled in the container from the multiple types of the information stored in the storage unit and controls the actuator based on the acquired information.

16. The droplet measurement system according to claim 14, wherein the acquisition unit includes:
an imaging unit that is placed such that a field of view is directed to a tip of the nozzle and a region extending vertically downward from the tip;
an imaging control unit that causes the imaging unit to execute imaging; and
an image processing unit that acquires a plurality of images, in a temporal sequence order, based on image data output from the imaging unit and that calculates the dripping cycle of the droplets dripping from the nozzle based on the plurality of images.

17. The droplet measurement system according to claim 16, wherein the image processing unit calculates the dripping cycle of the droplets dripping from the nozzle by detecting, from the plurality of images, a liquid discontinuity image that is an image that has captured therein the state immediately after the droplet separated from the tip.

18. The droplet measurement system according to claim 17, wherein the imaging control unit sets the field of view of the imaging unit to a rectangular region long in a vertical direction and causes the imaging unit to execute imaging, and
wherein the image processing unit acquires the plurality of images by generating images, in a temporal sequence order, that correspond to the rectangular region and that have captured therein the tip of the nozzle and the region extending vertically downward from the tip based on the image data output from the imaging unit.

19. The droplet measurement system according to claim 18, wherein the image processing unit includes a volume calculation unit that: acquires the liquid discontinuity image and a predetermined number of images following the liquid discontinuity image from the plurality of images; and that calculates the volume of the droplet dripping from the nozzle using the liquid discontinuity image and the predetermined number of images.

20. The droplet measurement system according to claim 19, further comprising a sensor that detects a tilt of the nozzle with respect to an axis in the vertical direction,
wherein the image processing unit further includes a correction unit that performs a correction operation for the volume of the liquid based on the tilt detection result by the sensor.

21. The droplet measurement system according to claim 19, wherein the image processing unit further includes a correction unit that retains a reference value of dimensions of an image of the nozzle in an image corresponding to the rectangular region and that performs a correction operation for the volume of the liquid based on the reference value and dimensions of an image of the nozzle captured in the liquid discontinuity image or the predetermined number of images.

22. The droplet measurement system according to claim 16, wherein an imaging frame rate of the imaging unit is 100 frames per second or more.

23. The droplet measurement system according to claim 18, wherein a ratio of the length in the vertical direction in the rectangular region to the length in a horizontal direction thereof is from 1.5 to 4.5, inclusive.

24. The droplet measurement system according to claim 16, further comprising:
a light source that is provided opposite to the imaging unit and that illuminates at least the tip of the nozzle and the region extending vertically downward from the tip; and
a filter that controls light distribution of light output from the light source.

25. The droplet measurement system according to claim 16, wherein the imaging unit further includes a telecentric lens.

26. The droplet measurement system according to claim 14, wherein the acquisition unit includes:
a sensor that counts the number of droplets dripping from the nozzle; and
an arithmetic unit that calculates the dripping cycle from the number of droplets counted by the sensor.

27. A droplet measurement method used in a droplet measurement system, the droplet measurement system comprising:
a nozzle that is provided to an infusion device that performs infusion of liquid filled in a container via a drip tube and that drips a droplet consisting of the liquid in the drip tube;
an actuator that varies a flow rate of the liquid by varying a pressing force with respect to a tube that causes the liquid accumulated in the drip tube to flow therethrough by driving a clamp that is provided on the tube in a pressable manner; and
a storage unit that stores therein information that is acquired in advance and that represents correlation between the volume of the droplet and a dripping cycle of droplets, the droplet measurement method comprising;
acquiring the dripping cycle of the droplets dripping from the nozzle; and
controlling the actuator such that the flow rate of the liquid falls within a predetermined range based on the information stored in the storage unit and the dripping cycle of the droplets dripping from the nozzle.

28. A computer readable recording device that stores thereon a droplet measurement program to be executed by a computer in a droplet measurement system, the droplet measurement system comprising:
a nozzle that is provided to an infusion device that performs infusion of liquid filled in a container via a drip tube and that drips a droplet consisting of the liquid in the drip tube;
an actuator that varies a flow rate of the liquid by varying a pressing force with respect to a tube that causes the liquid accumulated in the drip tube to flow therethrough by driving a clamp that is provided on the tube in a pressable manner; and
a storage unit that stores therein information that is acquired in advance and that represents correlation between the volume of the droplet and a dripping cycle of the droplet, the droplet measurement program causes the computer to:
acquire the dripping cycle of the droplets dripping from the nozzle; and
control the actuator such that the flow rate of the liquid falls within a predetermined range based on the information stored in the storage unit and the dripping cycle of the droplets dripping from the nozzle.
